(19) United States
(12) Patent Application Publication
(10) Pub. No.: US 2026/0283886 A1
(43) Pub. Date: Sep. 24, 2026

(54) SURGICAL TABLE WITH AUTOMATED KNOWLEDGE OF ATTACHED COMPONENTS

(71) Applicant: American Sterilizer Company, Mentor, OH (US)

(72) Inventors: John M. Kasunich, Mayfield Heights, OH (US); James A. Petrucci, Bluffton, SC (US); Michael E. Koerth, Newbury, OH (US); Breese J. Watson, Rocky River, OH (US); David Jesurun, South Euclid, OH (US)

(73) Assignee: American Sterilizer Company, Mentor, OH (US)

(21) Appl. No.: 19/479,473

(22) PCT Filed: May 14, 2024
(86) PCT No.: PCT/US2024/029239

§ 371 Date: Oct. 28, 2025

Related U.S. Application Data
(60) Provisional application No. 63/466,501, filed on May 15, 2023.

(51) Int. Cl. A61G 13/12; G16H 40/67

(52) U.S. Cl. CPC A61G 13/1285; G16H 40/67; A61G 2205/60

(57) ABSTRACT

A surgical table including attachable components that have pre-stored parameter data on-board the attachable component, wherein the parameter data is associated with the attachable component. Attachable components that are attached to the main platform of the surgical table communicate the pre-stored parameter data to the table controller via wired or wireless communications. In response, the table controller operates the surgical table with enhanced control feature in accordance with the parameter data.

### CLAIM OF PRIORITY AND CROSS-REFERENCE TO RELATED APPLICATIONS

This application claims priority to U.S. Provisional Patent Application Ser. No. 63/466,501, filed on May 15, 2023, entitled “SURGICAL TABLE WITH AUTOMATED KNOWLEDGE OF ATTACHED COMPONENTS,” which is incorporated by reference herein in its entirety.

### FIELD OF THE INVENTION

The present invention relates generally to surgical tables and the like, and more particularly to a surgical table having automated knowledge of parameters associated with an attachable component mechanically connected thereto.

### BACKGROUND OF THE INVENTION

Surgical tables typically have multiple attachable components for adapting the surgical table for a variety of different clinical needs. Some common attachable components include head and leg sections that are removably attachable to a main platform of the surgical table comprised of permanently-mounted back and seat sections.

In some prior art surgical tables, the table control system for operating the surgical table does not have knowledge of which, if any, attachable components are currently attached thereto. Other prior art surgical tables have a table control system that can simply detect the presence or absence of an attachable component. For example, the table control system relies on conventional contact limit switches or non-contact proximity switches to detect the presence/absence of attachable components. In both cases, the table control system has insufficient information about the specific parameters associated with the attachable components (e.g., length, width, height and thickness dimensions, weight, range of motion, etc.) to provide advanced control features for operation of the surgical table. With knowledge of specific parameters associated with an attached component, the table control system can respond differently based upon such parameters. For example, if the table control system has knowledge of the specific dimensions of an attachable head or leg section, then algorithms can be utilized by the table control system to provide a more robust collision avoidance feature during operation of the surgical table.

One disadvantage of the above-described prior art surgical tables is that the table control system operations are constrained by false warnings and/or the need to apply overly conservative limits to surgical table operations (e.g., range of motion limits) due to the need to program table control operations according to “worst-case” parameter data associated with compatible attachable components. As a result, the performance of known surgical tables can be further optimized.

Surgical tables are known in the prior art that have attachable components which solely transmit to the table control system a unique identifier corresponding to that type of attachable component. The table control system uses this unique identifier obtained from the attachable component to retrieve specific parameters associated with the attachable component from a data storage device of the table control system. For example, a pre-stored data table in the table control system's data storage device is used to correlate the unique identifier received from the attachable component with specific detailed parameters associated with the component.

One drawback to such prior art surgical tables is that the table control system itself must include pre-stored data for correlating unique identifiers of multiple attachable components with the specific parameters of the attachable components. As a result, when a new attachable component (i.e., a newly-available compatible component) is attached to the surgical table, there is a need to update the pre-stored data of the table control system with the unique identifier for this new attachable component along with its associated parameters. Therefore, such prior art surgical tables are not easily adapted for use with new attachable components, and may require frequent updates to the pre-stored data of the table control system in order to maintain compatibility with newly available attachable components.

The present invention provides a surgical table that overcomes the drawbacks of the prior art described above.

### SUMMARY OF THE INVENTION

In accordance with the present invention, there is provided a surgical table comprising a main platform including a seat section and a back section; at least one component that is attachable to the main platform, said component having a data storage element for storing parameter data associated with the component; and a table controller for receiving the parameter data from the component when the component is attached to the main platform, said main controller providing enhanced control features in accordance with the received parameter data.

In accordance with another aspect of the present invention, there is provided a method of operating a surgical table, said method comprising: receiving parameter data from a component attached to the surgical table; and providing enhanced control features of the surgical table in accordance with the received parameter data.

An advantage of the present invention is the provision of a surgical table that can automatically provide enhanced control features based upon received parameter data stored on-board attachable components that are attached to the surgical table, without the need for user input.

Another advantage of the present invention is the provision of a surgical table having one or more attachable components that have on-board storage of parameter data associated with the attachable component.

Yet another advantage of the present invention is the provision of a surgical table that includes attachable components that transmit parameter data associated with the component to a table controller of the surgical table, said parameter data being specific to the attached component, thereby eliminating the need for the table controller to store data for associating an identification designator of the attachable component with corresponding parameter data.

Yet another advantage of the present invention is the provision of a surgical table that provides maximum control functionality and a user experience that is not compromised by false warnings and overly conservative limits on operation of the surgical table.

A still further advantage of the present invention is the provision of a surgical table, wherein the mechanical action of attaching a component to the table also establishes an electrical connection therebetween.

These and other advantages will become apparent from the following description of illustrated embodiments taken together with the accompanying drawings and the appended claims.

### BRIEF DESCRIPTION OF THE DRAWINGS

The invention may take physical form in certain parts and arrangement of parts, embodiments of which will be described in detail in the specification and illustrated in the accompanying drawings which form a part hereof, and wherein:

FIG. 1 is a perspective view of a surgical table according to an exemplary embodiment of the present invention;

FIG. 2 is an exploded view of the surgical table, including a main platform comprised of seat and back sections, and attachable leg and head sections;

FIG. 3 is a distal end view of the seat section showing mechanical and control interfaces;

FIG. 4 is an end view of the leg section attachable component of the surgical table, showing mechanical and control interfaces;

FIG. 5 is a perspective view of the mechanical and control interfaces shown in FIG. 3;

FIG. 6 is a perspective view of the mechanical and control interfaces shown in FIG. 4;

FIG. 7 shows a hand-held primary user control of the surgical table, including keypad and display unit; and

FIG. 8 is a block diagram of the control system of the surgical table according to an embodiment of the present invention.

### DETAILED DESCRIPTION OF THE INVENTION

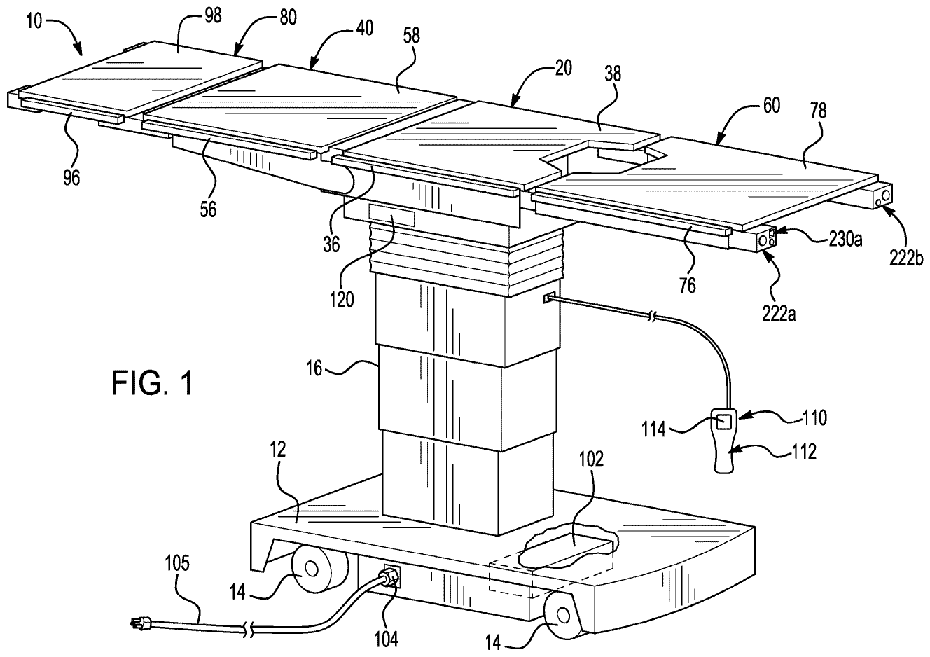

Referring now to the drawings wherein the showings are for the purposes of illustrating an embodiment of the invention only and not for the purposes of limiting same, FIG. 1 shows a representative surgical table 10 according to an embodiment of the present invention. It should be appreciated that surgical table 10 illustrates one embodiment of the present invention, and surgical tables taking alternative physical forms may be similarly adapted according to the present invention. In the illustrated embodiment, surgical table 10 includes a base 12; a column portion 16; and a main platform comprised of a moveable seat section 20 permanently mounted to column portion 16, and a moveable back section 40 permanently mounted to seat section 20.

Base 12 has a plurality of wheels 14 to facilitate movement of surgical table 10, and engageable/disengageable floor locks (not shown) to lock the position of surgical table 10. A power system 100 including a rechargeable battery 102 and a mains power receptacle 104 for connecting surgical table 10 to a mains power source via a power cord 105, are housed within base 12. In the illustrated embodiment, base 12 also houses a table controller, described below, that provides overall system control of surgical table 10.

Column portion 16 includes a lift system (not shown) for adjusting the height of surgical table 10. In this regard, the lift system adjusts the height of the main platform of surgical table 10 (i.e., seat section 20 that is mounted to column portion 16, and back section 40 that is permanently mounted to seat section 20).

A primary user control 110 in the form of a wired handheld control unit is connected to the table controller via a wired connection in the illustrated embodiment, but may also be connected to the table controller via a wireless connection. As best seen in FIG. 7, primary user control 110 includes a keypad 112 for user control inputs and a display 114 that provides surgical table information to the user, including a configured table image 116, which is discussed in further detail below. The illustrated embodiment of surgical table 10 also includes a secondary user control 120 mounted to the main platform that is electrically connected to the table controller. In one example, the secondary user control 120 includes a keypad substantially the same as keypad 112 of primary user control. In another example, the secondary user control 120 may include a display substantially the same as display 114 of primary user control.

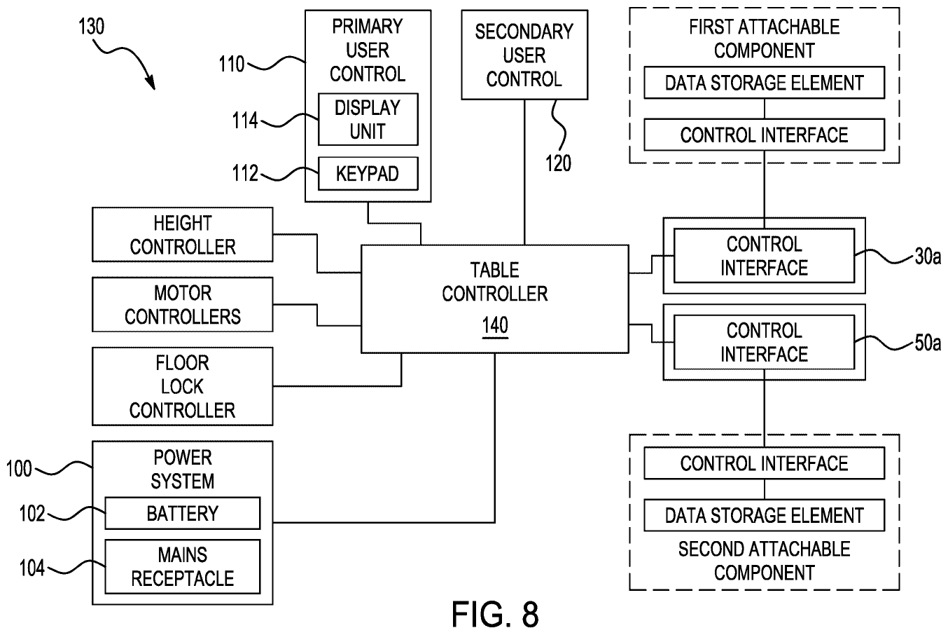

A general description of control system 130 of surgical table 10 according to an embodiment of the present invention will now be described with reference to FIG. 8 before providing a detailed description of seat section 20 and back section 40. Control system 130 includes a table controller 140 for providing overall system control of surgical table 10. In the illustrated embodiment, table controller 140 comprises a processing unit and a memory device. Control system 30 further comprises a height controller for controlling the height of column portion 16, a plurality of motor controllers for controlling motors that move table 10 to various desired positions (e.g., movement of trend, tilt, up/down, head slide, slide foot, back up, back down, leg up, leg down, left leg, right leg, flex, beach chair, kidney up, and kidney down), a floor lock controller for locking the floor position of table 10, a power system 100 comprised of rechargeable battery 102 and mains power receptacle 104 for connection to mains power, primary user control user 110, and secondary user control 120. In the illustrated embodiment, control system 130 also includes one or more control interfaces, described in detail below, which are used for communication with respective control interfaces of attachable components. In the illustrated embodiment, the first and second attachable components shown in FIG. 8 respectively represent leg section 60 and head section 80, which are described below.

It should be appreciated that FIG. 8 shows control system 130 as adapted for two (2) attachable components solely for the purpose of illustrating an embodiment of the present invention, and not for limiting same. However, it is contemplated that control system 130 may be adapted for one or more attachable components.

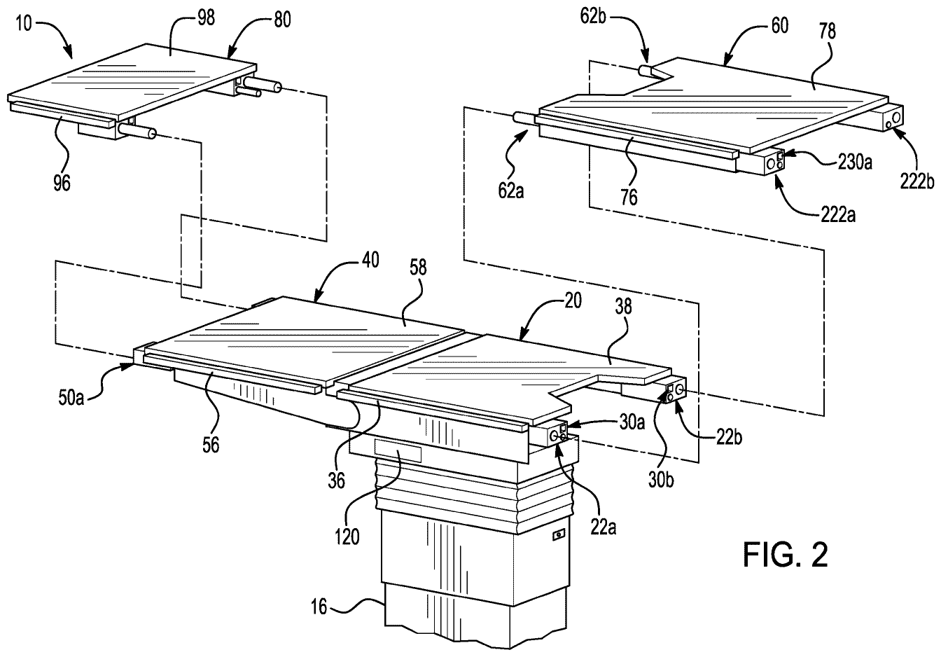

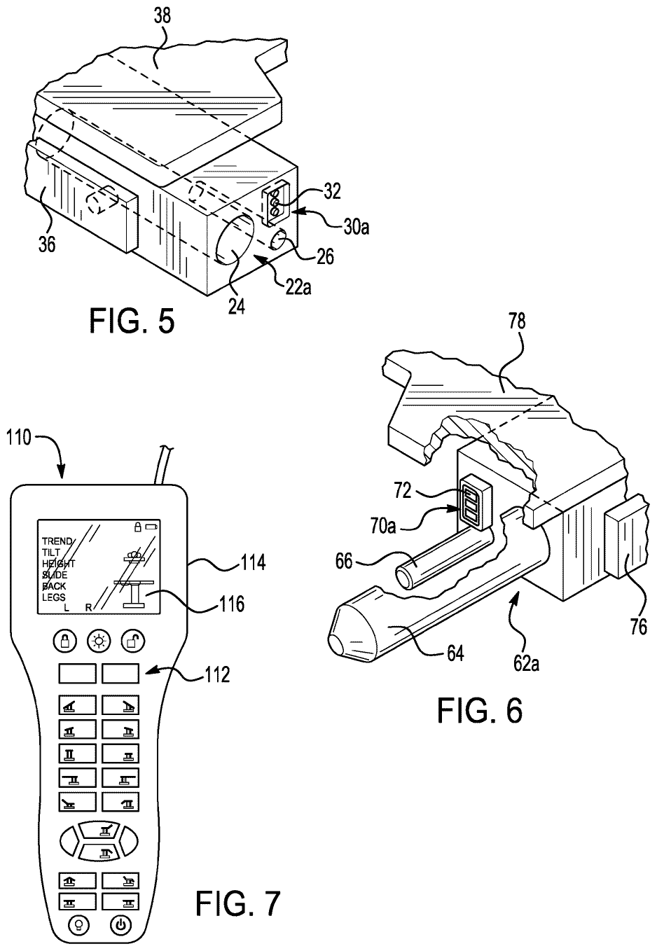

Returning now to FIG. 1, seat section 20 is comprised of a support surface 38 and a pair of lateral side rails 36 for mounting of attachable components. As best seen in FIGS. 2, 3 and 5, seat section 20 also includes a pair of mechanical interfaces 22a and 22b located at the free distal end thereof. Mechanical interfaces 22a and 22b are comprised of the mechanical elements for connecting an attachable component (described below) to the distal end of seat section 20. In the illustrated embodiment, mechanical interfaces 22a and 22b are each generally comprised of a socket 24, and a latch receptacle 26.

Furthermore, seat section 20 includes at least one control interface at the distal end thereof for communication with table controller 140. In the illustrated embodiment, seat section has two control interfaces 30a, 30b. Accordingly, this illustrated embodiment of the present invention is adapted to allow connection of different attachable components at each mechanical interface 22a, 22b. For example, the two attachable components could take the form of a right leg support section adapted for connection at mechanical interface 22a, and a left leg support section adapted for connection at mechanical interface 22b. In an alternative embodiment of the present invention where only one attachable component is to be attached to seat section 20, the second control interface can be omitted or deactivated.

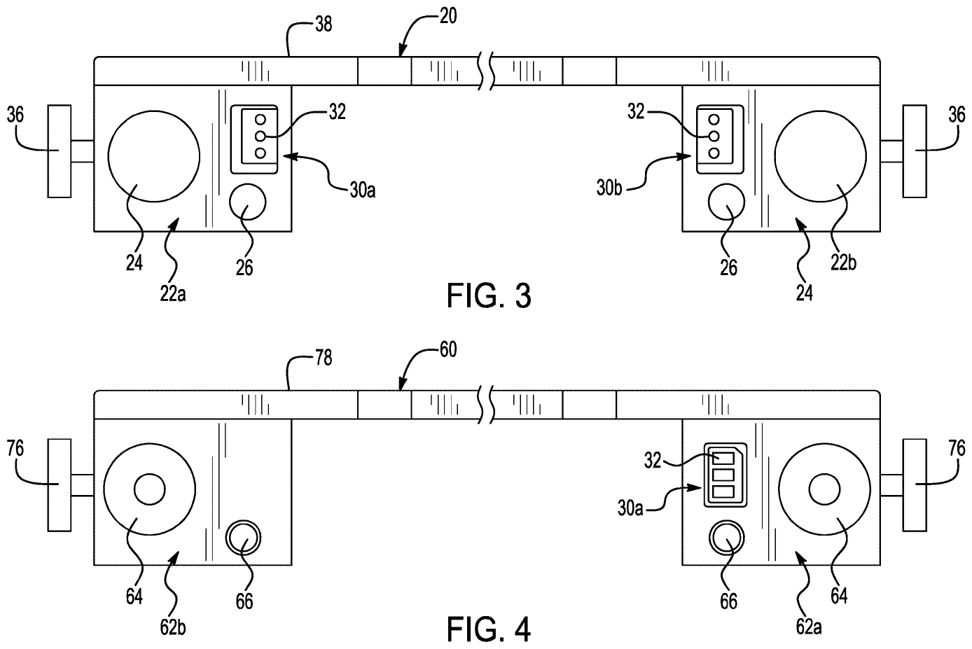

As best seen in FIG. 3, control interfaces 30a and 30b of the illustrated embodiment include one or more electrical contact members 32 in the form of pins, and electrical wiring (not shown) for electrically connecting contact members 32 with table controller 140.

It should be appreciated that the illustrated mechanical interfaces 22a, 22b and control interface 30 are shown solely for the purpose of describing an embodiment of the present invention, and not for limiting same. In this regard, it is contemplated that the mechanical and control interfaces may take alternative forms in accordance with the invention described herein.

Referring now to FIGS. 1 and 2, back section 40 includes a support surface 58 and a pair of lateral side rails 56. The free distal end of back section 40 includes mechanical interfaces that are substantially the same as mechanical interfaces 22a, 22b of seat section 20, and a control interface 50a (see FIGS. 1 and 8) that is substantially same as control interface 30a of seat section 20.

Surgical table 10 is adapted for attaching a wide range of compatible attachable components. Such attachable components may take a variety of forms, including, but not limited to: a leg section (e.g., standard leg section, left leg support section, and right leg support section), a head section (e.g., standard headrest, cone-shaped headrest, narrow headrest, dual-articulating headrest, and horseshoe headrest), stirrups, bariatric width extenders, bariatric foot extender, arm support sections, as well as other conventional attachable components used with surgical tables.

In the illustrated embodiment of the present invention, there are respectively shown first and second attachable components in the form of a leg section 60 attachable to seat section 20, and a head section 80 attachable to back section 40. Leg section 60 and head section 80 are described in further detail below.

Leg section 60 is comprised of a support surface 78 and pair of lateral side rails 76. As best seen in FIGS. 2, 4, and 6, leg section 60 also includes a pair of mechanical interfaces 62a and 62b located at a first end thereof. Mechanical interfaces 62a and 62b are comprised of mechanical elements for attaching leg section 60 to the distal end of seat section 20 described above. In the illustrated embodiment, mechanical interfaces 62a and 62b are each generally comprised of an elongated post 64 dimensioned to be received by mating socket 24 of seat section 20, and an elongated locking pin 66 dimensioned to be received by latch receptacle 26 of seat section 20. The illustrated post/socket arrangement is shown as an example configuration for providing a secure mechanical connection. It is contemplated that numerous alternative mechanical configurations may be substituted for the illustrated post/socket arrangement to provide a secure mechanical connection.

Furthermore, leg section 60 also includes a control interface 70a at the first end thereof for electrical connection with control interface 30a of seat section 20. In the illustrated embodiment, control interface 70a includes a one or more electrical contact members 72 in the form of pads that provide conductive surface areas. Contact members 72 are electrically connected with a data storage element (not shown) for storing parameter data associated with leg section 60. The data storage element may take a variety of different forms, including, but not limited to, a non-volatile memory (NVM) device such as a flash memory, an EEPROM, an EPROM, a conventional read-only memory (ROM), and the like.

Head section 80 will now be briefly described with reference to FIGS. 1 and 2. Head section 80 is comprised of a support surface 98 and a pair of lateral side rails 96. In addition, head section 80 includes mechanical interfaces and a control interface 50a that are substantially the same as mechanical interfaces 62a, 62b and control interface 70a of leg section 60, and thus, will not be described in further detail.

In accordance with the present invention, the data storage element of an attachable component stores parameter data associated therewith, including, but not limited to: (a) an identification designator that uniquely identifies the attachable component; (b) compatibility requirements of the attachable component for use with a particular surgical table; (c) dimensional information of the attachable component, including size (e.g., length, width, height, and thickness dimensions) and shape; (d) weight information of the attachable component; (e) location of center of gravity of the attachable component, and (f) safety information for the attachable component, such as allowable load.

It should be appreciated that the mechanical interfaces 62a, 62b and control interface 70a, as described herein, are shown solely for the purpose of illustrating an embodiment of the present invention, and not for limiting same.

The above-described control interfaces 30a and 70a illustrate an embodiment of wired communications interfaces for communicating parameter data to table controller 140. However, it is contemplated that the wired communication interfaces may take alternative forms in accordance with the present invention. For example, the wired communications interfaces may take the form of a pair of mating USB connectors using the standard USB communications protocol. In this alternative embodiment, a female USB connector is electrically connected with the data storage element of leg section 60, while a male USB connector housed in seat section 20 is electrically connected with table controller 140. Other conventional wired communications interfaces are also contemplated.

In addition to wired communications interfaces, it is further contemplated that the present invention may be implemented using a wireless communications interfaces with proximity sensing. In one such alternative embodiment, control interface 70a takes the form of a wireless transmitter and control interface 30a takes the form of a wireless receiver. Wireless communication protocols for Bluetooth, near-field communication (NFC), radio frequency identification (RFID), or the like, may be used for communication of parameter data from the attachable component to main controller 140 (e.g., between control interfaces 70a and 30a). It should be appreciated that a wireless communications interface may be particularly advantageous for use with attachable components that are connected to surgical table 10 by attachment of the component to the side rails described above (e.g., arm extensions, width extensions, stirrups, and like attachable components).

In embodiments where the control interface of the attachable component requires power, it may be provided by a long-life battery located in the attachable component itself. Alternatively, power may be provided to the attachable component from power system 100 of surgical table 10 (i.e., rechargeable battery 102 and/or mains power).

According to one contemplated alternative embodiment of the present invention, there is provided two-way communications between table controller 140 and the attachable component. With two-way communications, it is possible for table controller 140 to read data from and write data to a writable data storage element of the attachable component. For example, in such embodiment table controller 140 is programmed to read and increment a usage counter stored in the data storage element of the attachable component each time that the component is attached to a surgical table. This allows table controller 140 to monitor usage of the attachable component (by reading the usage counter upon attachment of a component) and alert a user of a maintenance interval (e.g., periodic inspection) based on the value of the usage counter. It should be appreciated that two-way communication is available with a wired communications interfaces, and with some wireless communications interfaces, such as NFC.

According to yet another alternative embodiment of the present invention, the data storage element of the attachable component takes the form of an optical code, such as a printed barcode, a QR code, or the like. The parameter data associated with the attachable component is encoded in the printed optical code. In this embodiment, the printed optical code eliminates the need for a control interface. In this regard, table controller 140 receives the parameter data using an optical reading device compatible with the optical code printed on the attachable component (e.g., a conventional bar code or QR code scanner).

A general description of the operation of surgical table 10 according to the illustrated embodiment of the present invention will now be described with reference to attachment of a first attachable component, in the form of a leg section 60, to seat section 20 of the main platform. Leg section 60 is aligned with seat section 20 such that posts 64 and locking pins 66 of mechanical interfaces 62a, 62b are respectively received into sockets 24 and latch receptacles 26 of mechanical interfaces 22a, 22b. As a result, leg section 60 makes a secure mechanical connection to seat section 20.

Once a secure mechanical connection is made, control interface 70a of leg section 60 makes an electrical connection with control interface 30a of seat section 20. In the illustrated embodiment, contact members 72 (pads) electrically connect with contact members 32 (pins), thereby allowing table controller 140 to obtain the parameter data stored in the data storage element of leg section 60. Table controller 140 is programmed to use the parameter data received from leg section 60 (and any other attached components) to provide enhanced control features for surgical table 10. Exemplary enhanced control features, include, but are not limited to, (1) collision avoidance behavior based on the dimensional information (i.e., size and shape) of the one or more attached components (e.g., limit the range of motion); (2) stability assessment and response based on the weight and location of the center of gravity of the one or more attached components, (3) selectively enabling/disabling various control features based on the parameter data associated with the one or more attached component, and (4) updating configured table image 116 shown on display unit 114 (FIG. 7) to display an accurate representation of the one or more attachable components currently attached surgical table 10. Table controller 140 updates configured table image 116 as components are attached and detached from the main platform.

In an embodiment where wireless communication interfaces are used for communicating parameter data from the attachable component to table controller 140, table controller 140 is programmed to enter a “search” mode following secure mechanical connection of the component, whereby table controller 140 searches for the presence of attachable components that are in close proximity. Using the wireless communication protocol, the attachable component transmits the parameter data that is received by table controller 140.

In accordance with yet another alternative embodiment of the present invention, an attachable component may include additional mechanical and control interfaces to allow a daisy chained configuration of attachable components. For example, in this embodiment, leg section 60 shown in FIGS. 1 and 2 includes additional mechanical and control interfaces at the second end of leg section 60 that is opposite the end having mechanical interfaces 62a, 62b and control interface 70a. Leg section 60 includes a pair of mechanical interfaces 222a and 222b (similar to mechanical interfaces 22a, 22b of seat section 20) for mechanically connecting an attachable component to leg section 60, and a control interface 230a (similar to control interface 30a of seat section 20) that is electrically connected with control interface 70a. As discussed above, control interface 70a is electrically connected with control interface 30a of seat section 20 in the configuration shown in FIG. 1.

Parameter data of a component secured to leg section 60 passes through control interface 230a to control interfaces 70a and 30a in a daisy chained configuration. For example, head section 80 can alternatively be attached to the second end of leg section 60 via mechanical interfaces 222a, 222b. Parameter data from head section 80 is then transmitted to table controller 140 via a communication pathway comprised of control interfaces 230a and 30a of leg section 60.

The foregoing describes specific embodiment(s) of the present invention. It should be appreciated that these embodiments are described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

What is claimed is:

1. A surgical table comprising:
a main platform including a seat section and a back section;
at least one component that is attachable to the main platform, said component having a data storage element for storing parameter data associated with the component; and
a table controller for receiving the parameter data from the component when the component is attached to the main platform, said main controller providing enhanced control features in accordance with the received parameter data.

2. The surgical table according to claim 1, wherein the parameter data includes one or more of the following:
(a) an identification designator that uniquely identifies the component;
(b) compatibility requirements of the component for use with the surgical table;
(c) dimensional information of the component;
(d) weight information of the component;
(e) location of center of gravity of the component; and
(f) safety information for the component.

3. The surgical table according to claim 2, wherein said dimensional information includes one or more of the following: size, length, width, height, thickness, and shape.

4. The surgical table according to claim 1, wherein the enhanced control features include one or more of the following:
(1) collision avoidance behavior based on the dimensional information of the component;
(2) stability assessment and response based on the weight information and the location of the center of gravity of the component,
(3) selectively enabling/disabling control features based on the parameter data; and
(4) updating a configured table image shown on a display unit to display an accurate representation of the components currently attached to surgical table.

5. The surgical table according to claim 1, wherein the data storage element is a non-volatile memory (NVM) device.

6. The surgical table according to claim 1, wherein the component and the main platform of the surgical table include respective control interfaces for electrically connecting the data storage element of the component with the table controller when the component is attached to the main platform of the surgical table.

7. The surgical table according to claim 1, wherein the data storage element is electrically connected with the table controller via a wired communications interface when the component is attached to the main platform of the surgical table.

8. The surgical table according to claim 1, wherein the data storage element transmits parameter data to the table controller via a wireless communications interface.

9. The surgical table according to claim 8, wherein the component includes a wireless transmitter for transmitting the parameter data to a wireless receiver in communication with the table controller.

10. The surgical table according to claim 9, wherein the wireless transmitter and wireless receiver use one of the following for wireless communications protocols: Bluetooth, near-field communication (NFC), and radio frequency identification (RFID).

11. The surgical table according to claim 1, wherein the surgical table further comprises a user control that includes a display having a configured table image showing components attached to the main platform of the surgical table, said configured table image updated by the table controller as components are attached and detached from the main platform.

12. The surgical table according to claim 1, wherein at least one component is adapted to connect with another component in a daisy chain configuration, whereby the table controller receives respective parameter data from all components in the daisy chain configuration.

13. The surgical table according to claim 1, wherein said main platform includes a seat section and a back section, wherein said seat section and back sections include:
a mechanical interface for mechanically connecting a component thereto; and
a control interface for electrically connecting a component to the table controller.

14. The surgical table according to claim 13, wherein said component includes:
a mechanical interface for mechanically connecting the component to the main platform; and
a control interface for electrically connecting the component to the table controller.

15. The surgical table according to claim 1, wherein
said main platform comprises:
a seat section, wherein said seat section includes a mechanical interface for mechanically connecting a component thereto and a control interface for electrically connecting a component to the table controller, said component comprising:
a mechanical interface adapted to mate with the mechanical interface of the seat section; and
a control interface for electrically connecting with the control interface of the seat section.

16. The surgical table according to claim 1, wherein
said main platform includes a back section, wherein said back section includes a mechanical interface for mechanically connecting a component thereto and a control interface for electrically connecting a component to the table controller, said component comprising:
a mechanical interface adapted to mate with the mechanical interface of the seat section; and
a control interface for electrically connecting with the control interface of the seat section.

17. The surgical table according to claim 1, wherein said main platform includes at least one rail adapted for attaching a component to the surgical table, wherein wireless communications are used to transmit the parameter data to the table controller.

18. The surgical table according to claim 1, wherein said table controller increments a usage counter for the component.

19. A method of operating a surgical table, said method comprising:
receiving parameter data from a component attached to the surgical table;
providing enhanced control features of the surgical table in accordance with the received parameter data.

20. The method according to claim 19, wherein the parameter data received from the component includes one or more of the following:
(a) an identification designator that uniquely identifies the component;
(b) compatibility requirements of the component for use with the surgical table;
(c) dimensional information of the component;
(d) weight information of the component;
(e) location of center of gravity of the component; and
(f) safety information for the component.
